(19) United States
(12) Patent Application Publication
(10) Pub. No.: US 2007/0249957 A1
(43) Pub. Date: Oct. 25, 2007

(54) MAPPING SPINAL MUSCLE TONE

(76) Inventors: Patrick Gentempo, Oakland, NJ (US); Lee Brody, Somerville, MA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/736,742

(22) Filed: Apr. 18, 2007

Related U.S. Application Data
(60) Provisional application No. 60/793,208, filed on Apr. 19, 2006.

(51) Int. Cl. A61B 5/04

(52) U.S. Cl. 600/546

(57) ABSTRACT

Systems and methods for collecting, analyzing, and/or displaying EMG data in the paraspinal muscles, include generating normalized EMG data using reference EMG data. Also disclosed are novel EMG parameters that are useful for at least one of diagnosis, determining a course of treatment, and/or monitoring a patient's response to a course of treatment.

### CROSS-REFERENCE TO RELATED APPLICATION

This application claims the benefit of U.S. Provisional Application No. 60/793,208, filed Apr. 19, 2006, the disclosure of which is incorporated by reference.

### BACKGROUND OF THE INVENTION

1. Field of the Invention

The present disclosure is generally directed to the diagnosis of medical conditions, and more particularly, to systems and methods for acquiring and analyzing electromyography data.

2. Description of the Related Art

Surface electromyography (EMG) is the measurement of electrical activity generated within a muscle using non-invasive electrodes placed on the surface of the skin proximal to the muscle of interest. There are two primary surface EMG protocols: (1) static EMG measures the static tone of muscles of a stationary patient for example, using a scanner known in the art, or adhesive electrodes, and (2) dynamic EMG measures the time course of electrical activity of a patient that is generating voluntary muscle contractions, typically using adhesive electrodes.

### SUMMARY OF THE INVENTION

Systems and methods for collecting, analyzing, and/or displaying EMG data in the paraspinal muscles, include generating normalized EMG data using reference EMG data. Also disclosed are novel EMG parameters that are useful for at least one of diagnosis, determining a course of treatment, and/or monitoring a patient's response to a course of treatment.

Accordingly, some embodiments provide a system for paraspinal electromyography comprising: at least one electrode for detecting an electromyography signal; a data processing unit receiving the electromyography signal from the at least one electrode and comprising machine readable instructions for generating normalized data from the electromyography signal; and an output device for graphically displaying normalized data from the data processing unit. The machine readable instructions for generating normalized data from the electromyography signal comprise: determining the ratio of the sum of selected patient electromyography signal data to the sum of corresponding reference electromyography data; and multiplying each patient electromyography signal data value by the ratio. The patient electromyography signal data are selected by a method comprising: determining the number of patient electromyography signal data that satisfy a threshold criterion; selecting only the patient electromyography signal data that satisfy the threshold criterion if the number of patient electromyography signal data that satisfy the threshold criterion exceeds a user defined value; and selecting all of the patient electromyography signal data if the number of patient electromyography signal data that satisfy the threshold criterion does not exceed a user defined value.

Other embodiments provide a method for normalizing patient electromyography data comprising: determining the ratio of the sum of selected patient electromyography data to the sum of corresponding reference electromyography data; multiplying each patient electromyography data value by the ratio. The patient electromyography data are selected by at least the following steps: determining the number of patient electromyography data that satisfy a threshold criterion; selecting only the patient electromyography data that satisfy the threshold criterion if the number of patient electromyography data that satisfy the threshold criterion exceeds a user defined value; and selecting all of the patient electromyography data if the number of patient electromyography data that satisfy the threshold criterion does not exceed a user defined value.

Other embodiments provide a method for determining a pattern analysis score of electromyography data comprising: determining the difference between a patient electromyography data value and a corresponding reference value for each patient electromyography data value; and averaging the differences.

Other embodiments provide a method for determining a pattern smoothness score of electromyography data comprising: (i) determining ratios between successive reference electromyography data values; (ii) selecting a starting actual patient electromyography data value corresponding to a starting reference electromyography data value; (iii) determining an expected successive electromyography data value for a successive patient electromyography data value from the starting patient electromyography data value and the ratio between the starting reference electromyography data value and successive reference electromyography data value; (iv) determining the difference between the expected successive electromyography data value and the actual successive patient electromyography data value; (vi) repeating at least once steps (ii)-(iv) for successive actual patient electromyography data values; and (vii) summing the difference determined in step (iv).

Other embodiments provide a method for determining a symmetry score of electromyography data comprising: determining the difference between two electromyography data values from a segment of a patient; and averaging the differences from a plurality of segments.

Other embodiments provide a method for determining a total energy of electromyography data comprising determining the ratio of the sum of selected patient electromyography data to the sum of corresponding reference electromyography data.

Other embodiments provide a method for determining a spasticity index of electromyography data comprising: collecting time-series electromyography data at a segment; transform time-series electromyography data into electromyography power density spectral data; and determine stability of electromyography power density spectral data over a data collection period.

Other embodiments provide a method for determining a spectral index of electromyography data comprising: collecting time-series patient electromyography data at left and right sides of a segment; transforming time-series patient electromyography data into patient electromyography power density spectral data; normalizing patient electromyography power density spectral data to reference electromyography power density spectral data; determining differences between median frequencies of normalized patient electromyography power density spectral data and median frequencies of reference electromyography power density spectral data; and averaging the differences.

### BRIEF DESCRIPTION OF THE DRAWINGS

FIG. 1 schematically illustrates an embodiment of a system for collecting and analyzing EMG data.

FIG. 2 is a flowchart illustrating an embodiment of a method for normalizing EMG data to reference data.

FIGS. 3A and 3B illustrate an embodiment of a continuous or “analog” graphical display of normalized EMG data.

FIGS. 4A and 4B illustrate a typical comparison of patient data to reference data known in the art, in which the patient data is color-coded based on the comparison to reference data.

FIG. 5 is a flowchart illustrating an embodiment of a method for determining a pattern analysis score from EMG data.

FIG. 6 is a flowchart illustrating an embodiment of a method for determining a pattern smoothness score from EMG data.

FIG. 7 is a flowchart illustrating an embodiment of a method for determining a symmetry score from EMG data.

FIG. 8 is a flowchart illustrating an embodiment of a method for determining a total energy from EMG data.

FIG. 9 is a flowchart illustrating an embodiment of a method for determining a spasticity index from EMG data.

FIG. 10 is a flowchart illustrating an embodiment of a method for determining a spectral index from EMG data.

### DETAILED DESCRIPTION OF THE PREFERRED EMBODIMENT

Systems and methods for collecting and displaying EMG data are disclosed below in the context of providing improved display and/or analysis of EMG data with reference to drawings. The systems and methods are described in this context because they have particular utility in this context. However, the systems and methods disclosed herein can be used in other contexts.

Surface EMG (SEMG) is useful in determining activation timing of the muscle(s), estimating the force produced by the muscle(s), determining an index of the rate at which a muscle fatigues, and diagnosing, for example, soft tissue injuries and/or vertebral subluxations. SEMG is a non-invasive technique using electrodes placed on the surface of the skin proximal to the muscle(s) of interest.

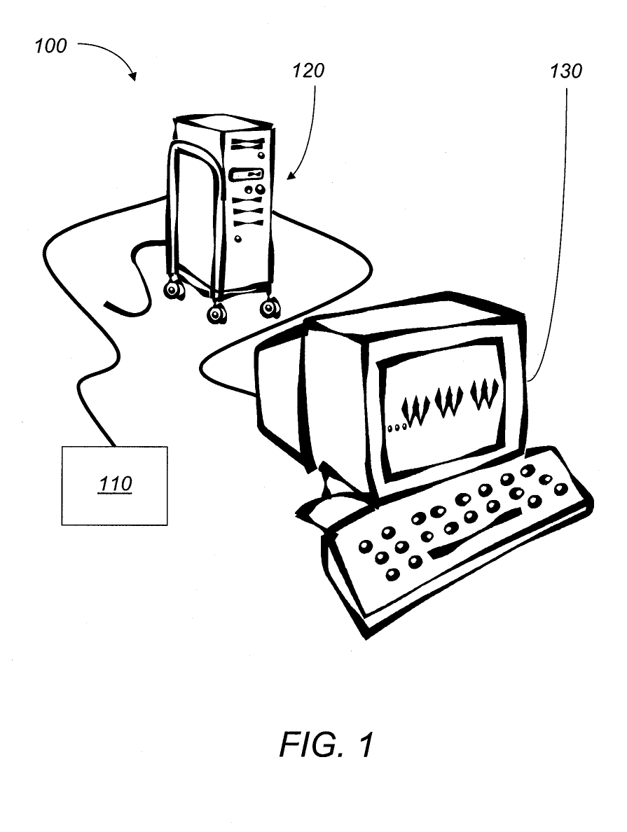

FIG. 1 illustrates schematically a system for paraspinal electromyography (EMG) comprising at least one electrode 110, a data processing unit 120 that receives EMG signals from the electrode 110, and an output device 130 for graphically displaying data from the data processing unit 120. The electrode 110 is any type of EMG electrode known in the art. In some preferred embodiments, the electrode 110 is a surface electrode of any type known in the art. Surface electrodes are applied to the surface of a patient's skin, typically proximal to the muscle(s) of interest. In some embodiments, the surface electrode is an adhesive electrode of any type known in the art. In other embodiments, the electrode is an electrode of an EMG scanner. Typically, an EMG scanner is a hand-held device comprising one or more electrodes on a surface thereof. The user contacts the electrodes with a patient's skin, whereupon EMG data is recorded. In some embodiments, a conductive material and/or gel is applied to at least a portion of the patient's skin prior to scanning. In some embodiments, the EMG scanner comprises a means for initiating data collection, for example, a trigger, button, and/or switch. Suitable EMG scanners are known in the art and are commercially available, for example, from the Chiropractic Leadership Alliance (Mahwah, N.J.). The EMG data is static or dynamic. Static EMG is recorded on a stationary patient, for example, using a scanner known in the art and/or using adhesive electrodes, as discussed above.

EMG data is collected using the electrode(s) 110. Some embodiments use a plurality of electrodes 110, for example, positioned at predetermined positions on a patient's back. Other embodiments use a scanner comprising one or more electrodes 110 which is sequentially moved to predetermined positions on a patient's back. In some preferred embodiments, the electrode 110 is a component of a hand-held scanner. In some embodiments, the EMG data is collected from a plurality of locations on a patient's back. For example, in some preferred embodiments, EMG data is collected in pair-wise sets, i.e., bilaterally, on a patient's back. In some preferred embodiments, a plurality of bilateral EMG measurements are taken, for example, at predetermined location on a patient's back. In some preferred embodiments, 15 bilateral EMG measurements are taken, one pair each at C1, C3, C5, C7, T1, T2, T4, T6, T8, T10, T12, L1, L2, L5, and S1. Each of these bilateral loci is also referred to generically as a “segment.” Those skilled in the art understand that other combinations of locations can be used in other embodiments. In some preferred embodiments, the data are static EMG data.

The data processing unit 120 is of any type known in the art, for example, a personal computer, a microcomputer, and/or a device comprising a microprocessor. As discussed above, the data processing unit 120 is configured to receive the output of the electrode(s) 110. In some preferred embodiments, the data processing unit 120 is configured to automatically execute at least some of the methods described herein. Accordingly, the data processing unit 120 comprises instructions for at least some of the disclosed methods in machine readable format. As discussed below, the data processing unit 120 also includes suitable hardware and/or software, for example, a graphics card and appropriate drives, for outputting graphical data to an output device 130. In some preferred embodiments, the data processing unit comprises other components known in the art, for example, volatile memory, non-volatile memory, data storage, networking devices, sound output devices, and/or other types of input devices, for example, keyboards, pointing devices, mice, microphones, cameras, combinations thereof, and the like.

The output device 130 comprises any type of output device known in the art, for example, a video display, a video projector, a CRT, a printer, and combinations thereof. In some preferred embodiments, the output device 130 is a video display, for example, a cathode ray tube (CRT) or liquid crystal display (LCD).

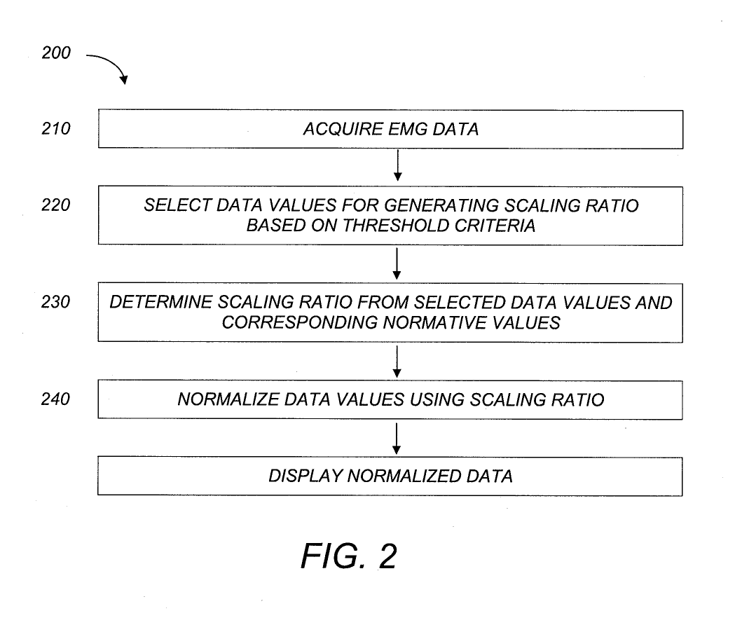

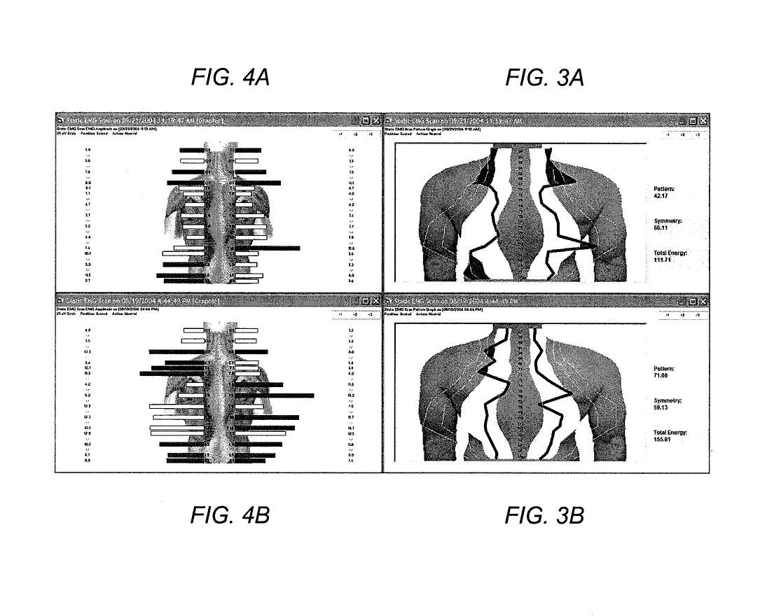

FIG. 2 illustrates a flow chart of an embodiment of a method 200 for analyzing EMG data. The method 200 is described with reference to the system illustrated in FIG. 1 as well as with reference to FIGS. 3A and 3B, and FIGS. 4A and 4B.

In step 210, EMG data is acquired, for example, using an electrode 110. As noted above, this step can be accomplished in various ways.

In step 220, the EMG data collected in step 210 are normalized using reference data using the data processing unit 120. In some embodiments, reference data is collected from a population of individuals. Other embodiments use reference data known in the art, for example, C, Kent & P. Gentempo “Normative data for paraspinal surface electromyographic scanning using a 25-500 Hz bandpass” Vertebral Subluxation Research 1996, 1(1):43, which provides EMG data for 15 bilateral segments at C1, C3, C5, C7, T1, T2, T4, T6, T8, T10, T12, L1, L2, L5, and S1. Other reference data is used in other embodiments. Preferred embodiments of the reference data include both means and standard deviations for EMG at each paraspinal location. In preferred embodiments, patient EMG data acquired from a particular location or segment is normalized against reference data taken from the same location or segment. For example, patient EMG data collected at the T1 segment are normalized against reference T1 values. The normalized patient data is also referred to herein as “normalized data.”

In some preferred embodiments, normalization is performed as follows: ratio between the sum of the patient EMG data values and the sum of the corresponding reference values is determined, which is also referred to herein as the scaling or normalizing ratio. Each of the patient EMG data values is multiplied by the scaling ratio to provide normalized data values.

In some embodiments, normalization is performed using a threshold-based algorithm, which eliminates outlier data in determining the scaling ratio. For example, in some preferred embodiments, the number of patient EMG values that fall below a threshold, for example, 1σ, are first determined. If that number is above a predetermined value, patient EMG values above the threshold value are ignored in calculating the scaling ratio. Otherwise, all patient EMG values are used in determining the scaling ratio. Those skilled in the art will understand that the threshold need not be a standard deviation. For example, in some embodiments, the threshold is an absolute value, a value relative to a reference value, or the like.

In the following example, the threshold value is 1σ and the predetermined value is 20 for 15 bilateral pairs of patient EMG data (30 total EMG values). In this example, the bilateral EMG data are collected at C1, C3, C5, C7, T1, T2, T4, T6, T8, T10, T12, L1, L2, L5, and S1. Where up to 20 of the EMG values are at or below the threshold value, the EMG values above the threshold are ignored in calculating the scaling ratio. Where fewer than 20 of the EMG values are at or below the threshold, all of the patient EMG values are used in calculating the scaling ratio. Those skilled in the art will understand that other thresholds and predetermined values can be used in other embodiments. Those skilled in the art will also understand that other embodiments can use other normalization methods.

In step 230, the normalized data is displayed on the output device 130. In some embodiments, the normalized data is displayed or overlaid over an image of a back (i.e., a posterior view of a human torso), for example, as illustrated in FIGS. 3A and 3B. In preferred embodiments, the normalized data is displayed as a continuous function as illustrated in FIGS. 3A and 3B, rather than as discrete levels and/or histograms as illustrated in FIGS. 4A and 4B. In some preferred embodiments, the displayed data is color coded with the colors indicating deviations from the reference data. For example, in FIGS. 3A and 3B, the normalized data are preferably color coded according to the standard deviation from the corresponding reference values: yellow is more than 1σ below the reference value, white is from 1σ below to 1σ above the reference value, green is from 1-2σ above the reference value, blue is from 2-3σ above the reference value, and red is greater than or equal to 3σ above the reference value. Different color codings are, of course, possible in other embodiments.

As discussed above, in some embodiments the resulting normalized data are displayed as a continuous “analog” mapping of normalized EMG values spine. Some embodiments use interpolation to determine EMG values at locations between those where EMG measurements were taken. In some embodiments, the interpolation is performed prior to normalization. In other embodiments, the normalized data are interpolated. For example, referring to FIG. 4A the EMG values for C2 are interpolated from the EMG values of C1 and C3.

Some embodiments of the analog mapping of normalized data can provide an improved view of a patient's overall EMG pattern, for example, as illustrated in FIGS. 3A and 3B. Accordingly, embodiments of the analog mapping of normalized data are also referred to as a “pattern graph.” Because the overall EMG pattern is typically difficult to extract from the discrete representations of each EMG level, some embodiments of the analog mapping are more useful in the clinical evaluation of certain conditions, for example, to identify those regions that deviate from the reference data, and to quantify their deviation.

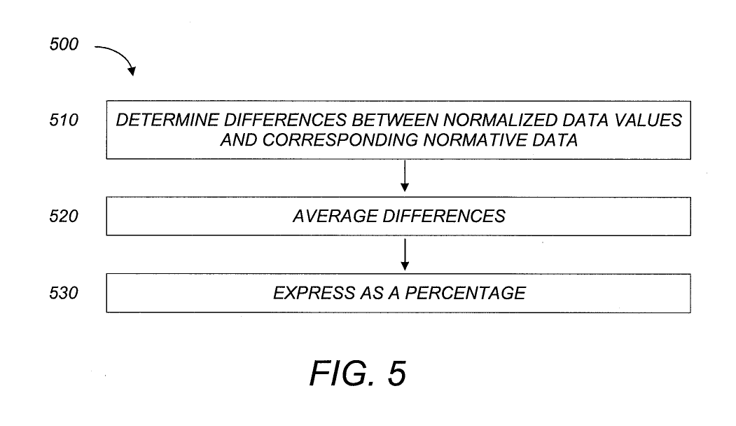

Also provided is an EMG parameter for an entire spinal scan referred to herein as a “pattern analysis score,” which quantifies the similarity between the pattern or shape of the muscle energy distribution a patient's EMG pattern and a reference data set. Embodiments of the pattern analysis score quantify the distribution of the bioelectric energy along the paraspinal muscles. Based on the reference data, the expected EMG pattern has less energy in the cervical region, more energy in the thoracic region, and less energy in the lumbar region. In some embodiments, the pattern analysis score is expressed as a number between 1-100, with 100 being a perfect match to the reference data. Those skilled in the art will understand that the pattern analysis score is expressible in other ways, for example, as a deviation from the reference data, where a lower score indicates a lower deviation. In some embodiments, the pattern analysis score is determined according to an embodiment of a method 500 illustrated as a flowchart in FIG. 5. In step 510, the difference between each of the normalized EMG values and the corresponding reference EMG values is determined. In step 520, these differences are averaged. In step 530, the average from step 520 is expressed as a percentage and subtracted from 100 to provide the pattern analysis score. In some embodiments, the pattern analysis score is displayed on the output device 130.

Also provided is an EMG parameter referred to herein as “pattern smoothness score,” which quantifies the shape of a patient's EMG pattern compared with the shape of a reference data set. Embodiments of the pattern smoothness score quantify the similarity of the transitions from each level to the next of the patient data to that of the reference data. From a clinical standpoint, it is expected that the muscle energy distribution transitions smoothly between adjacent levels, which is observed in reference EMG data. Embodiments of a reference EMG pattern are smooth, that is, there are gradual increases and decreases in muscle tone along the spine. In patients with some chronic conditions, the pattern is less smooth, with jagged and/or abrupt increases and/or decreases in tone along the spine. In some cases, the smoothness improves during the course of care.

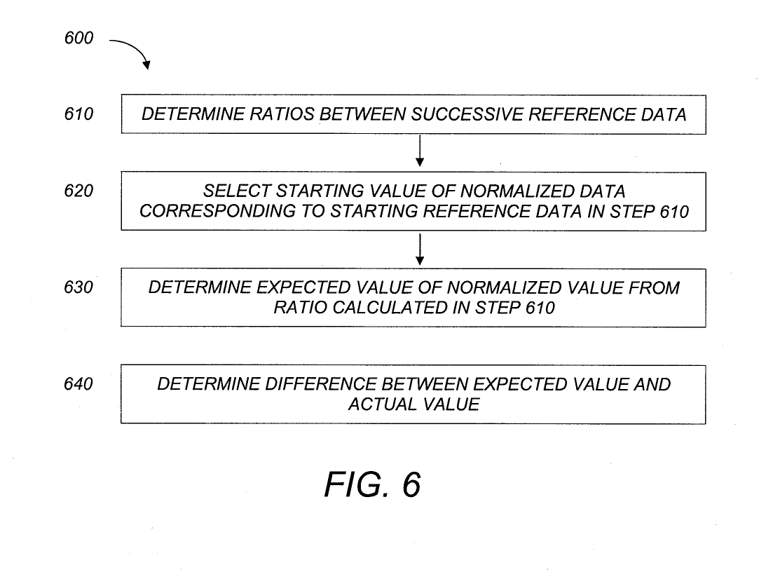

An embodiment of a method 600 for calculating a pattern smoothness score is illustrated as a flow chart in FIG. 6. In step 610, the ratios between successive values in the reference data are determined. In step 620, the starting value of the normalized data that corresponds to the starting value of the reference data used in step 610 is determined. In step 630, the expected value of the next value of normalized data is determined by multiplying the normalized value by the appropriate ratio of reference data calculated in step 610. In step 640, the difference between the expected value and the actual value is determined. In some embodiments, the difference is expressed in μV or as a percentage. Other embodiments use other methods to determine the difference in step 640. In step 650, steps 630 and 640 are repeated for the remaining normalized values using the actual normalized values as the starting values. In step 650, the sum of the differences is determined. In some embodiments, the score is determined by expressing the sum from step 650 as a percentage and subtracting from I 00. In some embodiments, a smoothness scores are independently calculated for the right side normalized EMG data and left-side normalized EMG data.

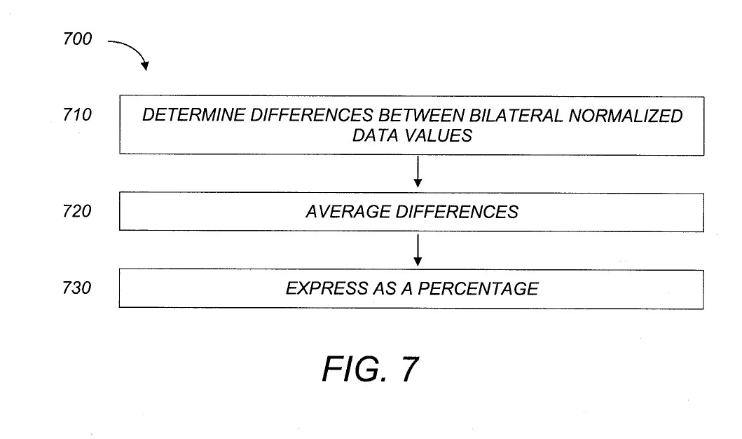

Some embodiments provide an EMG parameter for an entire spinal scan referred to herein as a “symmetry score,” which quantifies the left-right balance of the EMG data, thereby reflecting the left-right balance in the muscle energy down the full spine. In the reference data, these muscles are pulling left and right with equal force at each level of the spine. In some embodiments, the symmetry score is expressed as a number from 1-100 with 100 being a perfect symmetry score. An embodiment of a method 700 for calculating the symmetry score is illustrated as a flowchart in FIG. 7. In step 710, the difference between each pair of bilateral normalized data is calculated. In step 720, the average of these differences is calculated. In step 730, the average is expressed as a percentage and subtracted from 100 to provide a symmetry score. Those skilled in the art will understand that the symmetry score is expressible in other ways, for example, as a deviation from the reference data. Those skilled in the art will understand that, in some embodiments, the symmetry score is calculated using the unnormalized EMG data instead of the normalized data. In some embodiments, the symmetry score is displayed on the output device 130.

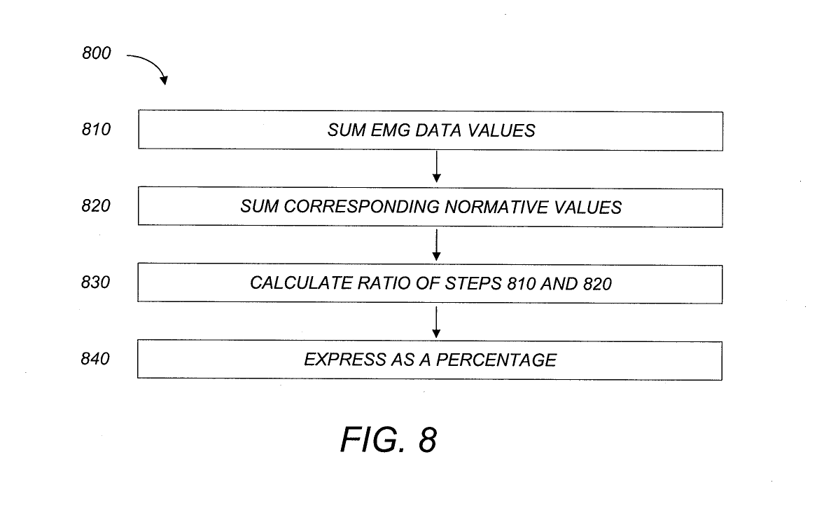

Some embodiments provide an EMG parameter referred to herein as “total energy,” which quantifies the total energy of the EMG scan compared to the reference data. In embodiments in which the total energy is based on the normalized data, it provides a comparison of overall energy in a patient's EMG scan compared with the reference data. In some embodiments, the total energy is as a number of 1-100+, with 100 being an ideal score. In some embodiments, the total energy can be above 100. An embodiment of a method 800 for calculating a total energy is illustrated as a flowchart in FIG. 8. In step 810, the unnormalized EMG data values are summed. In step 820, the reference EMG values are summed. In step 830, the ratio between the patient EMG data values and the reference EMG values is calculated. In step 840, the ratio is expressed as a percentage by multiplying by 100. In some embodiments, the total energy is displayed on the output device 130.

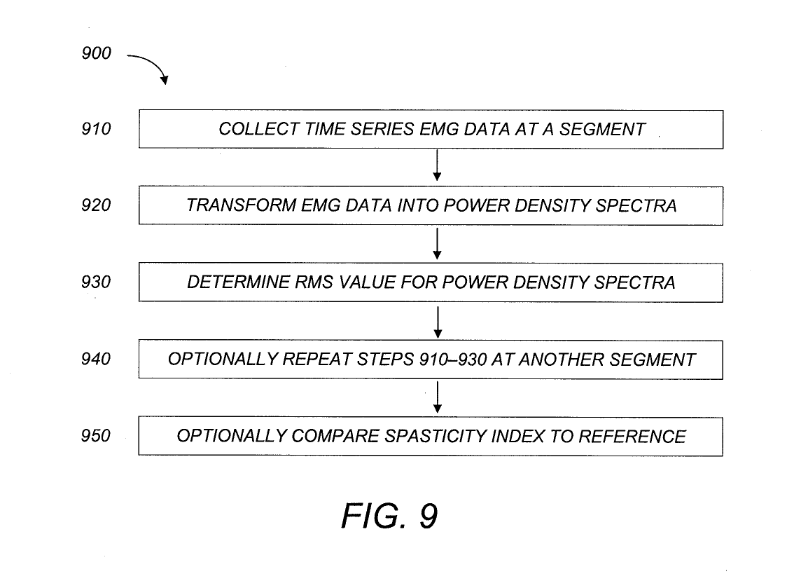

Some embodiments provide an EMG parameter referred to herein as a “spasticity index,” which quantifies the stability of the muscle tone at each segment by monitoring the stability of the EMG data signal in both the time and frequency domains as the measurement is taken. The spasticity index provides a range of stability of muscle tone along the muscles of the spine, which is clinically significant because muscles because certain clinical conditions do not result in abnormal EMG patterns, but exhibit a lack of stability in the static muscle tone. In some embodiments, the spasticity index is determined by method 900 illustrated in a flowchart in FIG. 9. The illustrated embodiment uses frequency domain data. Those skilled in the art will understand the application of the method 900 to time domain data.

In step 910, time series EMG data is collected at a segment after it is determined that the electrode(s) are properly placed and the signal is valid. In some embodiments, the data are collected for a predetermined time, for example, 3 seconds. In some embodiments, the data is collected in a static scan, that is, without voluntary contraction of the musculature. Typically, the EMG signal is typically band-limited from 20-500 Hz. In step 920, the EMG data are transformed into power density spectra (PDS). In some embodiments, a power density spectrum is determined for a predetermined data collection time. In some embodiments, the power density spectrum is determined periodically. For example, in some embodiments, the power density spectrum is calculated for every 0.5 sec of data, and the power density spectrum updated every 0.1 sec. Three seconds of data results in 30 EMG PSDs.

In step 930, the stability of the EMG output is determined for the data collection period. In some embodiments, an RMS value is calculated for each EMG PSD, and these values compared. In other embodiments, the stability is determined by tracking the stability of the spectral shapes with time in the EMG PSDs. For examples, some embodiments monitor the median frequency of the EMG PSDs. Other embodiments use other criteria known in the art. In some preferred embodiments, the stability of the EMG output is expressed as the standard deviation of the median frequency of the EMG PSDs.

In optional step 940, a spasticity index is determined for another segment by repeating steps 910-930. In optional step 950, one or more of the patient's spasticity index data are compared with reference data. In some embodiments, the spasticity index is displayed on the output device 130.

Also disclosed herein are EMG parameters in which the EMG data are collected as time-series and optionally transformed, for example, as EMG power density spectrum (EMG PDS) data. These EMG parameters are generally referred to herein as “spectral parameters.” Some embodiments of the spectral parameters are similar to parameters discussed above that are determined from single time point EMG data, for example, the pattern graph, pattern analysis score, pattern smoothness score, symmetry score, and total energy. Those skilled in the art will recognize that there are many ways to quantify the characteristics of an EMG power spectrum, and many ways to quantify similarities and differences of two or more EMG power spectra.

In some embodiments, the EMG PDS data are collected as described above for in step 910 of method 900. Some embodiments of the spectral parameters use normalized PDS data. In some embodiments, the EMG PDS data are normalized against reference EMG PDS data to provide normalized PDS data, for example, by a method analogous to step 220 of method 200.

Briefly, the patient EMG PDS data acquired from each segment is normalized against reference data for which the median frequency and standard deviation is known. A scaling or normalizing ratio is calculated by summing the median frequencies of the patient EMG PDS data, and dividing by the sum of the median frequencies of the reference data. Some embodiments use a threshold-based algorithm, as discussed above, which avoids skewing of the scaling ratio by outlier data. In some embodiments, the normalized PDS data are graphically displayed, for example, overlaid on a image of a human back. In some embodiments, the data is displayed as analog data, referred to as a “spectral index graph.”

Some embodiments provided herein provide an EMG parameter referred to herein as a “spectral index,” which quantifies the spectral characteristics of the EMG signal at each segment. Some embodiments of the spectral index quantify the spectral content of paraspinal muscles at rest. The spectral index in normal muscles is different than that of the muscles in various clinical conditions, and it is believed that these differences are caused by differences in recruited muscle types, fatigue of the muscles, and the like.

Embodiments of the spectral index are determined by: (1) comparing the similarities of the EMG PDS data collected at different points within a patient; (2) comparing the similarities of each of the EMG PDS data collected in a patient to those of reference data; or (3) a combination of comparing the EMG PDS data collected within a patient as well as a comparison to reference data.

Some embodiments of the spectral index are determined analogously to the pattern analysis score described above using the normalized PDS data as the data input. In some embodiments, the EMG PDS is calculated from a 0.5 second sliding average of EMG data, and updated every 0.1 second. Some embodiments of the spectral index use a reference PDS. In some embodiments, the reference PDS is either single spectrum, for example, the last identified when the clinician chooses to accept the data, or is an average of several spectra, which are averaged by any method known in the art. In other embodiments, a reference PDS is compiled from PDS data acquired from a selected population.

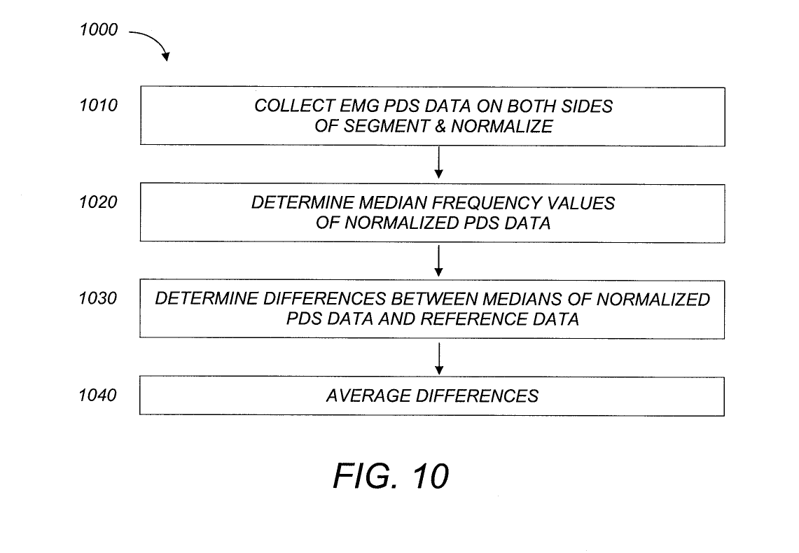

An embodiment of a method 1000 for determining a spectral index is illustrated as a flowchart in FIG. 10. In step 1010, PDS data are collected on left and right sides of one or more segments of interest along the paraspinal musculature, and normalized as discussed above. In step 1020, the median frequency of each normalized PSD data is determined. In step 1030, the differences between the median frequencies of each normalized PSD data and the median frequencies of the reference data is determined for each segment. In step 1040, the differences are averaged. In some embodiments, the average is expressed as a percentage and subtracted from 100 to provide a spectral index score.

Some embodiments provided herein provide an EMG parameter referred to herein as a “spectral symmetry,” which quantifies the overall differences of the EMG signal between the spectral characteristics of the left and right sides at each segment. In some embodiments spectral symmetry is calculated analogously to the symmetry score, comparing the median frequencies of the EMG PDS.

Certain of the methods are described using normalized EMG data, either time point or time series. Those skilled in the art will understand that other embodiments of one or more of the disclosed methods use unnormalized EMG data.

Those skilled in the art will understand that changes in the systems, devices, and processes described above are possible, for example, adding and/or removing components and/or steps, and/or changing their orders. Moreover, the systems, devices, and processes described herein are useful for other purposes, for example, the diagnosis, evaluation, and treatment of patients.

Moreover, while the above detailed description has shown, described, and pointed out novel features as exemplified in\various embodiments, it will be understood that various omissions, substitutions, and changes in the form and details of the systems, devices, and/or processes illustrated may be made by those skilled in the art without departing from the spirit of the invention. As will be recognized, some embodiments do not provide all of the features and benefits set forth herein, as some features may be used or practiced separately from others.

What is claimed is:

1. A system for paraspinal electromyography comprising:
at least one electrode for detecting an electromyography signal;
a data processing unit receiving the electromyography signal from the at least one electrode and comprising machine readable instructions for generating normalized data from the electromyography signal; and
an output device for graphically displaying normalized data from the data processing unit,
wherein the machine readable instructions for generating normalized data from the electromyography signal comprise:
determining the ratio of the sum of selected patient electromyography signal data to the sum of corresponding reference electromyography data; and
multiplying each patient electromyography signal data value by the ratio,
wherein the patient electromyography signal data are selected by a method comprising:
determining the number of patient electromyography signal data that satisfy a threshold criterion;
selecting only the patient electromyography signal data that satisfy the threshold criterion if the number of patient electromyography signal data that satisfy the threshold criterion exceeds a user defined value; and
selecting all of the patient electromyography signal data if the number of patient electromyography signal data that satisfy the threshold criterion does not exceed a user defined value.

2. A method for normalizing patient electromyography data comprising:
determining the ratio of the sum of selected patient electromyography data to the sum of corresponding reference electromyography data;
multiplying each patient electromyography data value by the ratio,
wherein the patient electromyography data are selected by at least the following steps:
determining the number of patient electromyography data that satisfy a threshold criterion;
selecting only the patient electromyography data that satisfy the threshold criterion if the number of patient electromyography data that satisfy the threshold criterion exceeds a user defined value; and
selecting all of the patient electromyography data if the number of patient electromyography data that satisfy the threshold criterion does not exceed a user defined value.

3. A method for determining a pattern analysis score of electromyography data comprising:
determining the difference between a patient electromyography data value and a corresponding reference value for each patient electromyography data value; and
averaging the differences.

4. A method for determining a pattern smoothness score of electromyography data comprising:
(i) determining ratios between successive reference electromyography data values;
(ii) selecting a starting actual patient electromyography data value corresponding to a starting reference electromyography data value;
(iii) determining an expected successive electromyography data value for a successive patient electromyography data value from the starting patient electromyography data value and the ratio between the starting reference electromyography data value and successive reference electromyography data value;
(iv) determining the difference between the expected successive electromyography data value and the actual successive patient electromyography data value;
(vi) repeating at least once steps (ii)-iv) for successive actual patient electromyography data values; and
(vii) summing the difference determined in step (iv).

5. A method for determining a symmetry score of electromyography data comprising:
determining the difference between two electromyography data values from a segment of a patient; and
averaging the differences from a plurality of segments.

6. A method for determining a total energy of electromyography data comprising determining the ratio of the sum of selected patient electromyography data to the sum of corresponding reference electromyography data.

7. A method for determining a spasticity index of electromyography data comprising:
collecting time-series electromyography data at a segment;
transform time-series electromyography data into electromyography power density spectral data; and
determine stability of electromyography power density spectral data over a data collection period.

8. A method for determining a spectral index of electromyography data comprising:
collecting time-series patient electromyography data at left and right sides of a segment;
transforming time-series patient electromyography data into patient electromyography power density spectral data;
normalizing patient electromyography power density spectral data to reference electromyography power density spectral data;
determining differences between median frequencies of normalized patient electromyography power density spectral data and median frequencies of reference electromyography power density spectral data; and
averaging the differences.
